(19) United States
(12) Patent Application Publication
(10) Pub. No.: US 2018/0142313 A1
(43) Pub. Date: May 24, 2018

(54) PROCESS FOR THE PREPARATION OF A FRUCTOSE-RICH SOLUTION FROM A SOLID COMPOSITION COMPRISING FRUCTOSE AND GLUCOSE

(71) Applicant: Synvina C.V., Amsterdam (NL)

(72) Inventors: Benjamin McKay, Amsterdam (NL); Gerardus Johannes Maria Gruter, Amsterdam (NL); Ilona Van Zandvoort, Amsterdam (NL)

(21) Appl. No.: 15/570,429

(22) PCT Filed: May 27, 2016
(86) PCT No.: PCT/NL2016/050376

§ 371 Date: Oct. 30, 2017

(30) Foreign Application Priority Data
May 27, 2015 (NL) 2014858

(51) Int. Cl. C13K 11/00; C13K 3/00; C07D 307/20; B01D 11/02

(52) U.S. Cl. CPC C13K 11/00; B01D 11/02; C07D 307/20; C13K 3/00

(57) ABSTRACT

A fructose-rich solution is prepared from a solid composition including fructose and glucose, in a process including i) admixing the solid composition including fructose and glucose with a selective solvent that consist for at least 80% wt of methanol to obtain a slurry of glucose-rich solids in a methanolic fructose-rich solution; and ii) separating the methanolic fructose-rich solution from the glucose-rich solids.

### CROSS-REFERENCE TO RELATED APPLICATIONS

This application is the National Stage of International Application No. PCT/NL2016/050376 filed May 27, 2016, which claims the benefit of Netherlands Application No. NL 2014858, filed May 27, 2015, the contents of which is incorporated by reference herein.

### FIELD OF THE INVENTION

The present invention relates to a process for the preparation of a fructose-rich solution from a solid composition comprising fructose and glucose.

### BACKGROUND OF THE INVENTION

It is well known that carbohydrates and in particular saccharides such as fructose and glucose are interesting bio-based compounds that form important feedstocks for a variety of chemicals. One such interesting development is the preparation of hydroxymethyl furfural or an ether or ester thereof for the production of biofuels or for the manufacture of furan dicarboxylic acid which is an important intermediate for the formation of plasticizers, pharmaceuticals, oligomers and polymers, such as polyesters, polyamides and polyurethanes. An example of the documents that describe the manufacture of ethers of hydroxymethyl furfural is U.S. Pat. No. 8,877,950. This document claims a continuous flow process for the manufacture of ethers of 5-hydroxymethylfurfural by reacting a fructose-containing starting material with methanol, in the presence of an acid catalyst at a temperature of 175 to 225° C. and at a residence time in the flow process from 1 to 10 minutes, wherein water is present as solvent in a ratio of alcohol/water from 50:1 to 10:1. The specification shows that the conversion of fructose gives higher yields than the conversion of glucose.

Since fructose provides better results than glucose, it is desirable to obtain feedstocks that consist mainly of fructose. However, glucose is more abundantly available. For instance, it is the monosaccharide that constitutes starch and cellulose. Therefore, processes have been developed to isomerize glucose to form fructose. Such isomerization can be achieved chemically or enzymatically. Since the conversion of glucose to fructose is equilibrium limited, the isomerization will result in a mixture of fructose and glucose. In commercial processes for the isomerization of glucose to fructose, the fructose content after isomerization is typically between 40 and 45%, based on the glucose starting material. Also the hydrolysis of sucrose results in a mixture of these two sugars. In this case the fructose to glucose ratio is 50:50. For further use there is a need to separate glucose from such mixtures of glucose and fructose in order to obtain fructose-enriched compositions. The separated glucose-rich fraction may be employed for other applications or may be recycled to an isomerization process. In this way fructose can be obtained in a more or less pure form.

Therefore, studies have been made to separate fructose from mixtures of fructose and glucose. In U.S. Pat. No. 3,533,839 a process is described wherein a mixture of solid glucose and fructose is treated with anhydrous absolute ethanol containing anhydrous calcium chloride to dissolve fructose as an anhydrous addition compound and leave glucose undissolved. When solid glucose has been separated from the solution, a hydrated addition product of calcium chloride and fructose is precipitated from the solution by the addition of water. In order to remove the fructose itself from the addition product, it is dissolved in nearly an equal amount by weight of water. The calcium chloride is removed either as such by means of a treatment with an ion exchange resin or by converting the calcium chloride into calcium sulfate followed by filtration of the calcium sulfate and removal of other ions by means of ion exchange resin treatment. It is evident that this process requires the use of anhydrous ethanol and anhydrous calcium chloride and subsequent complicated treatment of the hydrated addition product to free up fructose.

U.S. Pat. No. 3,812,010 describes a process wherein an aqueous solution of a blend of fructose and glucose is subjected to water evaporation and to addition of an alcohol, such as methanol or methyl glycol. The aqueous solution must be completely salt and acid-free. This can be achieved by a pre-treatment, such as treatment with an ion exchange resin. Allegedly selective crystallization of glucose and fructose is possible. The crystallization is stated to take three days. In an example sucrose is isomerized in water to give a solution of glucose and fructose. The solution was concentrated on a hot water bath in vacuum. Methanol was added to the concentrate to give a solution with 4.7% water. After seeding with glucose further solid glucose is formed in the course of three days. It is evident that in this example there is no separation between fructose and glucose from a solid composition. In another example a 1 kg of a dry mixture of fructose and glucose in a weight ratio of 48.5:51.5, is dissolved in 2 kg of water-free methanol whilst stirring and heating on a water bath. After cooling and seeding with glucose crystals the solution was stirred for 24 hours at 10° C. White glucose crystals were obtained from the solution in an amount of about 46%, based on the original amount of glucose.

Application of the differences of the solubility of fructose and glucose in ionic liquids has been described in U.S. Pat. No. 7,942,972. Ionic liquids of so-called type A, e.g. 1,3-dimethyl-imidazolium dimethyl phosphate, can be used to selectively dissolve glucose from a solid or liquid fructose/glucose blend. Ionic liquids of so-called type B, e.g. 1-ethyl-2-methyl-imidazolium ethyl sulfate, are the better solvents for fructose and can thus be used to leave glucose undissolved. In order to be able to re-use the ionic liquids, it is suggested to extract the dissolved fructose or glucose with water. However, the ionic liquids mentioned as examples, are miscible with water and therefore are unsuitable for recovery of the dissolved glucose or fructose by water extraction, and for the recycle of the ionic liquid.

### SUMMARY OF THE INVENTION

Hence there is a need for a simple manner to obtain enriched fructose from a solid composition that comprises fructose and glucose wherein the fructose can then be easily processed further. It has now been found that the use of a solvent that mainly consists of methanol can be used to obtain fructose-rich solutions from solid mixtures of fructose and glucose with dissolving only minor amounts of glucose. Accordingly, the present invention provides a process for the preparation of a fructose-rich solution from a solid composition comprising fructose and glucose, comprising
i) admixing the solid composition comprising fructose and glucose with a selective solvent that consist for at least 80% wt of methanol to obtain a slurry of glucose-rich solids in a methanolic fructose-rich solution; and
ii) separating the methanolic fructose-rich solution from the glucose-rich solids.

In this context the reference to glucose-rich solids refers to solids wherein the proportion of glucose is higher than in the solid composition comprising fructose and glucose that was admixed with the selective solvent, based on the amounts of fructose and glucose. Mutatis mutandis the reference to fructose-rich solution refers to the solution wherein the proportion of fructose is higher than in the solid composition comprising fructose and glucose that was admixed with the selective solvent, based on the amounts of fructose and glucose.

### DETAILED DESCRIPTION OF THE INVENTION

According to a journal article (Kiang et al., Phytochemistry, 7 (1968) 1035-1037) the root powder of a plant, i.e. Mikania Cordata (Burm.F.) B. L. Robinson, was extracted for one week with methanol. After concentration a residue was formed. The residue was filtered off, and the filtrate was further concentrated to give a gummy residue that was stirred with water. After filtration and extraction of the filtrate with ethyl acetate the remaining aqueous layer was concentrated to give a brown sticky mass, wherein glucose and fructose were identified. It is clear that this article does not establish that glucose and fructose were present in the root powder. It is generally known that inulin which consists of chain-terminating glucosyl moieties and repetitive fructosyl moieties, are typically found in roots. After extraction with methanol, evaporation of the methanol and stirring with water, the inulin may have hydrolyzed to glucose and fructose during these treatments. The disclosure does not relate to the selective dissolution of fructose from a fructose- and glucose-comprising solid composition wherein a slurry is prepared to form a fructose-rich solution.

The process according to the present invention has the advantage that the methanolic fructose-rich solution can be directly passed on to a conversion of fructose into an ether of HMF, if desired. If solid fructose is desired as the final product, it is relatively easy to precipitate fructose from methanol since its solubility is much lower in methanol than in water. Moreover, evaporation of methanol, if desired, is much easier and less energy-consuming than the evaporation of water. All these advantages are obtained by the simple partial dissolution of the solid composition comprising fructose and glucose in a methanol-containing selective solvent. The present process provides an advantage over the example of U.S. Pat. No. 3,812,010 in that there is no need to dissolve the solids of fructose and glucose completely, and then cool and seed to precipitate glucose-rich solids. The present process is also surprising in view of the teaching of U.S. Pat. No. 3,533,839 wherein it is taught that although the addition of 5 vol % methanol to the ethanol used can be quite harmless as to the yield of the fructose addition compound, the more methanol is used the less the yield becomes. It is stated that when a mixture of equal volumes of methanol and ethanol is used the yield is lowered from about 95% to about 60%. According to the present process fructose-containing solutions comprising fructose in a ratio of fructose to glucose of 4:1 can be obtained from solid compositions that have a major amount of glucose relative to fructose, e.g. a weight ratio of 2:1. Hence, the present invention enables enrichment to fructose by a factor in the range of 5 to 10.

The selective solvent consists for at least 80% wt, preferably for at least 85% wt of methanol, based on the weight of the solvent. That means that the selective solvent may contain other components. Such components include water, acids, e.g. those that have been used in the preparation of invert sugar, bases, e.g. those that have been used in the chemical isomerization of glucose to fructose, other alcohols, such as ethanol, and other organic diluents. Such other diluents may include liquids in which fructose and/or glucose are soluble or insoluble. Examples include sulfoxides, such as DMSO (dimethyl sulfoxide), ethers, such as diethyl ether, esters, such as methyl or ethyl levulinate, aldehydes such as formaldehyde or acetaldehyde and ketones, e.g. acetone. The process according to the present invention does not require that very pure methanol is used as selective solvent. Preferably, the amount of methanol in the selective solvent is at least 90% wt. The selective solvent may suitably comprise mainly methanol and water. Therefore, the selective solvent preferably comprises at most 10% wt of water. Since the solubility of fructose is enhanced by the presence of some water, whilst the glucose solubility hardly increases when the water content in methanol is relatively small, the selective solvent preferably comprises at least 1% wt water. More preferably, the selective solvent comprises at least 90% wt methanol and from 2 to 10% wt water, the percentages being based on the weight of the selective solvent. When the content of water and methanol do not add up to 100% wt, the balance may be provided by other diluents, such as those mentioned hereinabove. When the solid composition already contains some adsorbed water, it may not be desired to add additional water to the selective solvent. The amount of adsorbed water may already be sufficient to provide for a satisfactory solubility level of fructose. The selective solvent may then substantially consist of methanol. Such may also be the case when the solid composition is comprised in a small amount of water such that an aqueous slurry of solid particles is obtained.

Contrary to the procedure in U.S. Pat. No. 3,533,839 the resulting slurry of glucose-rich solids and methanolic fructose-rich solution does not need to be subjected to cooling and seeding and prolonged stirring and cooling in order to obtain glucose precipitate. In the process of the present invention the slurry of solids and solution may be subjected to separation without any further treatment. Therefore, the methanolic fructose-rich solution is preferably separated from the glucose-rich solids by means of one or more operations selected from the group consisting of sedimentation, filtration, centrifugation, flotation and decantation. Preferably, at least one of filtration and centrifugation is used as separation technique.

The methanolic fructose-rich solution may comprise glucose in addition to a major portion of fructose. If desired, at least part of the glucose that is contained in the methanolic fructose-rich solution may be separated therefrom, suitably by precipitation. Therefore, the methanolic fructose-rich solution may optionally be further cooled, and optionally stirred, to achieve crystallization of a glucose-rich precipitate. The precipitate thus obtained may comprise a certain amount of fructose. Dependent on parameters such as the temperature, the amount of solvent used, the content of methanol in the selective solvent and the solubility of the various components in the selective solvent, the amount of fructose contained in the precipitate may be significant. Therefore, the skilled person may refrain from doing so.

In order to obtain a methanolic fructose-rich solution that contains as much fructose as possible but also as little glucose as feasible the solid composition comprising fructose and glucose and the solvent are suitably admixed at a temperature of at most 50° C. When the temperature exceeds 50° C. not only fructose will dissolve in the solvent, but also significant amounts of glucose may get dissolved. The temperature should therefore be as low as feasible. However, when the temperature is below 0° C., the solubility of fructose in the solvent may become too low, so that impractical amounts of solvent are to be used to obtain a satisfactory content of fructose in the methanolic solution. Therefore, the temperature for the admixing of the solid composition with the selective solvent is suitably conducted at a temperature of at least 0° C., preferably at least 20° C. Hence, more preferably, the temperature is in the range of 20 to 40° C. The pressure does not play an important role in the process of the present invention. Although not preferred, the pressure may be elevated, e.g. up to 50 bar. When sub-atmospheric pressures are to be applied it is to be borne in mind that the combination of pressure and temperature should not be too low so that the methanol-comprising selective solvent will evaporate. Pressures around atmospheric, e.g. from 0.8 to 2 bar, appear to be most practical.

The amount of selective solvent may play a role in avoiding the dissolution of too much glucose. The amount of selective solvent should preferably be such that the fructose is dissolved completely and the remaining glucose-rich solids contain virtually no fructose. Suitably, the amount of selective solvent that is admixed with the solid composition is in the range of 0.1:1 to 10:1, preferably from 0.5:1 to 10:1, based on the weight of selective solvent per weight of solid composition. More preferably, the amount of selective solvent per amount of solid composition is in the range of 0.8:1 to 5:1, based on the weight of selective solvent per weight of solid composition. When the amount of selective solvent is below 0.1:1, a considerable amount of fructose may remain in the solid state and may form part of the glucose-rich solids. Alternatively, if the amount of selective solvent is above 10:1, glucose may be dissolved in a considerable amount and may form part of the fructose-rich solution. Moreover, when such large excesses of selective solvent are used, the resulting solution tends to be rather dilute with a low concentration of fructose. The recovery of fructose from such diluted solutions thus becomes more complex and expensive. Very good results were obtained when the amount of selective solvent and other conditions were selected such that the resulting methanolic fructose-rich solution has a fructose to glucose weight ratio in the range of 75:25 to 95:5. As explained above, other conditions that may play a role in the present invention may include the temperature of the admixing and the composition of the selective solvent.

The solid composition comprising fructose and glucose may have been obtained in a variety of methods. One way of producing the solid composition that is used in the present process is by hydrolyzing sucrose. The hydrolysis or inversion of sucrose is typically catalyzed by an acid. Instead of sucrose also honey or the hydrolysate of inulin can be used. Another way is by isomerizing glucose to fructose. This isomerization can be achieved in a chemical way or an enzymatic way, as indicated above. Starting materials for such isomerization include glucose and the glucose-containing hydrolysis products of cellulose and starch, and also of other glucose-comprising carbohydrates. Advantageously, the solid composition that is used in the present process has been derived from the isomerization of glucose to fructose. The solid composition comprises fructose and glucose. The content of these monosaccharides in the solid composition may vary. Evidently the proportion of components in the solid composition that also are able to be dissolved in the selective solvent is suitably as low as possible. In such a case the glucose-rich solids further comprise these insoluble components. In order to obtain products from the present process with a satisfactory purity, the solid composition preferably consists at least for at least 95% wt of fructose and glucose, based on the weight of the solid composition. The solid composition may comprise hydrated compounds. However, the composition is solid, i.e. it is different from a gas or liquid in that it retains its shape and density. That implies that the present process does not cover the use of a viscous syrup of fructose and glucose. Hence the solid composition suitably comprises up to 5% wt of water. However, solid compositions that are comprised as solid particles in a slurry are encompassed by the present process. Optional other components include disaccharides such as sucrose, and oligomers of saccharides from which fructose and glucose have been obtained. The liquid phase in such a slurry may comprise also some fructose and glucose. When the slurry is admixed with the selective solvent, the admixture of the slurry and the solvent will result in glucose-rich solids and a methanolic fructose-rich solution. The fructose in the methanolic fructose-rich solution may originate from the solid particles as well as from the dissolved fructose in the liquid phase of the slurry.

As the solid composition can be obtained in a variety of ways the proportions of the constituents in the composition can be very different, too. When the solid composition has been derived from the inversion of sucrose the amounts of fructose and glucose will be about equal. On the other hand if the solid composition is derived from the hydrolysate of inulin the proportion of fructose will be significantly higher than that of glucose. Even when the product of the isomerization of glucose is used, the fructose content can be lower than the content of glucose. Typically, the solid composition comprises fructose and glucose in a fructose to glucose weight ratio of 1:10 to 10:1. In practice solid compositions with a weight ratio of glucose to fructose of 50:50 to 80:20 are available.

In many of the methods to produce a solid composition that contains fructose and glucose an aqueous solution of fructose and glucose is obtained. The inversion of sucrose or honey and the hydrolysis of inulin take place in an aqueous environment. Also the enzymatic isomerization of glucose occurs in water. When the isomerization is conducted chemically, typically an aqueous alkaline solution is used. In order to obtain a solid composition from these solutions it is necessary to remove water therefrom. Accordingly, the solid composition of the present invention has suitably been obtained by the evaporation of water from a liquid saccharide solution, comprising water, fructose and glucose. In the art it has been realized that it is difficult to obtain glucose and fructose crystals from an aqueous syrup due to the high viscosity that is encountered during the cooling and evaporation of the aqueous syrup and the tendency to form a supersaturated solution. By means of seeding crystals it has been attempted to achieve or accelerate the crystallization of fructose and glucose in such syrups. In certain patents it has been disclosed that the addition of certain organic liquids are beneficial in obtaining solid fructose and glucose. In U.S. Pat. No. 4,643,773 ethanol and isopropanol are added to corn syrup having a solids content of 91.6% wt. When seed crystals are also added to the mixture thus obtained, solid fructose is obtained. In comparative tests this procedure was repeated with methanol and blends of methanol and ethanol. In U.S. Pat. No. 5,004,507 a process is described wherein a cooled fructose syrup is subjected to evaporation in an evaporator until crystals are formed. Then the cooled syrup is mixed with an alcohol and held over a cooling period of time sufficient to substantially complete the crystallization process in a holding step. Thereafter, the output of the holding step is recovered is filtered and dried and recovered as the end product. The solid composition used in the present process may be obtained as the product of these or similar processes.

The process according to the present invention preferably employs a solid composition that has been obtained from the evaporation of water from a liquid aqueous saccharides solution, wherein the evaporation has been achieved by mixing the aqueous saccharides solution comprising water, fructose and glucose, with a carrier liquid in which the saccharides are insoluble and that has a boiling point higher than that of water, and subjecting the mixture thus obtained to an evaporation step. By carrier liquid is understood a liquid compound that is chemically inert vis-à-vis the components in the aqueous saccharides solution, i.e. chemically inert towards water, fructose and glucose. It is envisaged that by using a carrier liquid it is less difficult to obtain a solid composition from the mixture of carrier liquid and saccharides than from a viscous syrup of saccharides and water. The viscosity of such syrups may get too high to allow for an effective solidification, whereas the solubility of saccharides in water is so high that it is virtually impossible to obtain solid saccharides from diluted solutions of saccharides in water.

The carrier liquid has a boiling point above that of water. In order to facilitate the separation of the carrier liquid from the water and the acid, the carrier liquid preferably has an atmospheric boiling point that is at least 60° C., more preferably 75° C., above that of water. Hence, the carrier liquid preferably has a boiling point of at least 160° C. In order to facilitate the separation even further the atmospheric boiling point of the carrier liquid is suitably at least 190° C. The upper limit of the boiling point range for the carrier liquid is not critical. The only requirement for the carrier liquid is that it is in the liquid phase when it is admixed to the aqueous saccharide solution. For instance, the boiling point of the carrier liquid may be as high as 350° C.

The carrier liquid is suitably selected from the liquids in which saccharides are insoluble. In this specification by ‘insoluble’ is understood that a substance in question dissolves for less than 1 g/100 mL at 25° C. The carrier liquid may be miscible with water, as long as saccharides are insoluble therein. When a carrier liquid is immiscible with water, saccharides tend to be insoluble in such a carrier liquid. Hence, the carrier liquid is preferably immiscible with water. In this specification by ‘immiscible’ is understood that when a volume of water and a volume of the carrier liquid are added to form a mixture the volumes of the liquid layers that emerge deviate less than 5% vol/vol from the volumes that were originally added to form the mixture.

An attractive type of carrier liquids is immiscible with water. Suitable carrier liquids are advantageously hydrocarbon oils. Examples of such oils are shale oil, refined petroleum oils, and refined paraffin oils. Also suitable oils are polyisobutylene and poly-alpha-olefins. Other branched polyolefins and other petroleum liquids with a sufficiently high boiling point can also be used. The oils are also characterized by their viscosity. Suitable oils have a kinematic viscosity in the range of 1 to 200 cSt (mm²/s) at 100° C., determined in accordance with ASTM D 445.

An alternative type of carrier liquid is miscible with water. One such carrier liquid is constituted by polymers and oligomers of alkylene glycol. The miscibility of such polymers with water depends on the chain length, both as to the number of alkylene oxide monomers and the number of carbon atoms in the alkylene oxide monomer itself. Already the dimer of ethylene glycol, having a boiling point of about 245° C. is a suitable carrier liquid. Hence, when the polyalkylene oxide is a polymer of ethylene oxide, the number of ethylene oxide units may suitably be as low as two. The higher polymers are usually characterized by their average molecular weight. Polyethylene oxides are prepared with very high molecular weights, e.g. up to 10,000,000. However, typically the upper limit of the molecular weight of the polyethylene oxide that may be used in the present process is generally about 25,000. In addition to polyethylene glycol, polymers of other alkylene oxides may be used, in particular polymers of alkylene oxides having 2 to 6 carbon atoms, such as polypropylene glycol, polybutylene glycol and mixtures thereof. Other suitable carrier liquids that are miscible with water are alkyl levulinates. Alkyl levulinates, such as methyl levulinate and ethyl levulinate, are known as fuel components; cf. U.S. Pat. No. 7,351,268. It has surprisingly been found that when alkyl levulinates are used as carrier liquids for aqueous saccharide solutions and are being used for the present process, they allow for evaporation of water and the solidification of fructose and glucose. Accordingly, the carrier liquid is preferably selected from the group consisting of alkylene glycols and alkyl levulinates. When alkyl levulinates are used, the alkyl group suitably has 1 to 6 carbon atoms. Preferably, the alkyl group is selected from methyl and ethyl.

The products of the process of the present invention are glucose-rich solids and a methanolic fructose-rich solution. The glucose-rich solids may be used as such, e.g. in the production of other bio-based chemicals, such as ethylene glycol. Alternatively, e.g. when the solid composition that is used as starting material for the present process is obtained from an isomerization of glucose, the glucose-rich solids are suitably recycled to the isomerization of glucose to fructose.

The methanolic fructose-rich solution may be subjected to a crystallization process as described in U.S. Pat. No. 4,643,773, to achieve the crystallization of fructose from this methanolic solution. Advantageously, at least part of the methanolic fructose-rich solution is used as feedstock for the preparation of the methyl ether of 5-hydroxymethylfurfural by reacting the fructose-rich solution, or the part thereof, as starting material with methanol in the presence of a heterogeneous or homogeneous acid catalyst at a temperature in the range of 150 to 300° C. It is preferred to have a relatively high concentration of fructose in the methanolic solution. Therefore, it is advantageous to contact the fructose in the methanolic solution with an acid catalyst at a temperature of at most 100° C., preferably in the range of 20 to 60° C. so that methyl fructosides are formed. The solubility thereof in methanol is higher than the solubility of fructose itself. Therefore, some methanol can be evaporated from the solution of fructose and fructosides to obtain an enriched solution. This enriched solution can conveniently be used as the starting material in the above-mentioned method for the preparation of the methyl ether of 5-hydroxymethylfurfural. The method for this preparation is known, e.g. from U.S. Pat. No. 8,877,950. The method has preferably a reaction temperature of 175 to 300° C., more preferably from 175 to 225° C. The acid catalyst may be a heterogeneous catalyst, e.g. selected from the group consisting of ion exchange resins, zeolites and combinations thereof. Alternatively, the acid catalyst is homogeneous, e.g. a mineral acid, such as nitric, hydrochloric or sulfuric acid. The contact time of the fructose-rich solution with the catalyst is advantageously from 0.5 to 15 minutes, preferably from 1 to 10 minutes. Although the method may be conducted as a batch process, it is preferred to conduct the method as a continuous flow process. The fructose-rich methanolic solution may comprise some water. That will especially be the case when the solution has been contacted with an acid catalyst at a temperature below 100° C. to form fructosides. Small amounts of water are not detrimental. On the contrary, it is preferred that the starting material comprises water, suitably in a weight ratio of methanol to water in the range of 50:1 to 2:1.

The process will be further illustrated by means of the following examples.

### Example 1

Certain amounts of solvent were mixed with 100 parts by weight (pbw) of a solid composition comprising 65 pbw glucose and 35 pbw fructose at 35° C. The solvent consisted of 95% wt methanol and 5% wt water. After a contact time of 30 or 60 minutes, during which the mixture of the solids and solvent was stirred, the liquid phase obtained was separated from the remaining solids by centrifugation. The fraction of fructose and the fraction of glucose, based on the sum of dissolved fructose and glucose, in the liquid phase were determined. Also the total concentration of hexose (=fructose and glucose), expressed as % wt of the liquid phase was analyzed. From these data the amount of fructose in the liquid phase compared to the original amount of fructose in the solid composition could be established. The results are shown in Table 1. Table 1 also provides the amount of solvent, in pbw.

TABLE 1

| | | | | | | Fructose |
|---|---|---|---|---|---|---|
| | Amount | Contact | Fruc- | | | in liquid |
| Exp. | Solvent, | time, | tose | Glucose | Concentration, | phase, |
| No. | pbw | min | fraction | fraction | % wt | pbw |
| 1 | 100 | 30 | 0.87 | 0.13 | 23 | 20 |
| 2 | 200 | 30 | 0.86 | 0.14 | 14 | 24 |
| 3 | 300 | 30 | 0.80 | 0.20 | 10 | 24 |
| 4 | 400 | 30 | 0.78 | 0.22 | 8 | 25 |
| 5 | 50 | 60 | 0.86 | 0.14 | 31 | 13 |
| 6 | 100 | 60 | 0.89 | 0.11 | 25 | 22 |
| 7 | 200 | 60 | 0.84 | 0.16 | 14 | 24 |
| 8 | 300 | 60 | 0.79 | 0.21 | 11 | 26 |
| 9 | 400 | 60 | 0.77 | 0.23 | 8 | 25 |

The above results show that the methanolic solvent can selectively dissolve fructose from a mixture of glucose and fructose. When the amount of methanolic solvent is greater than 50% wt, based on the weight of the mixture of glucose and fructose, a high proportion of the originally present fructose can be selectively recovered by selective dissolution in the methanolic solvent.

### Example 2

Different solid compositions having different relative amounts of fructose and glucose were used in a series of experiments wherein the same solvent was employed as the one used in Example 1. The temperature was 35° C., and the contact time was 60 min. In the experiments 100 pbw of solvent were contacted with 10 pbw of solid composition. The relative amounts of glucose and fructose (Glu/Fru) are shown in Table 2. Table 2 also includes the results of the experiments in the same manner as shown for Example 1.

TABLE 2

| | | | | | Fructose in |
|---|---|---|---|---|---|
| Exp. | Glu/Fru, | Fructose | Glucose | Concentration, | liquid |
| No. | w/w | fraction | fraction | % wt | phase, pbw |
| 10 | 75/25 | 0.85 | 0.15 | 22 | 19 |
| 11 | 70/30 | 0.87 | 0.13 | 25 | 22 |
| 12 | 65/35 | 0.88 | 0.12 | 27 | 24 |

The results show that when the solid composition contains relatively more fructose the concentration of fructose in the liquid phase also increases.

### Example 3

To show the effect of other components in the selective solvent varying amounts of methyl levulinate (ML) and water (H₂O) were added to methanol (MeOH) to make up the selective solvent. The resulting solvents were contacted with a solid composition comprising 65 pbw of glucose and 35 pbw of fructose. In each experiment 100 pbw of selective solvent was contacted with 100 pbw of solid composition at 35° C. for 60 min.

The results are shown in Table 3.

TABLE 3

| | | | | | | | Fructose |
|---|---|---|---|---|---|---|---|
| | Selective | | | Fruc- | | Con- | in liquid |
| Exp. | solvent, % wt | | | tose | Glucose | centration, | phase, |
| No. | ML | H₂O | MeOH | fraction | fraction | % wt | pbw |
| 13 | 10 | — | 90 | 0.83 | 0.17 | 13 | 11 |
| 14 | 2.5 | 2.5 | 95 | 0.85 | 0.15 | 20 | 17 |
| 15 | 5 | — | 95 | 0.83 | 0.17 | 12 | 10 |
| 16 | 5 | 5 | 90 | 0.85 | 0.15 | 19 | 16 |

The results show that the influence of methyl levulinate on the selectivity of the dissolution is marginal. However, it is apparent that the presence of water benefits the overall solubility of fructose in the resulting selective solvent. In all cases the solvent selectively dissolved fructose.

### Example 4

To show a method to obtain a solid composition of fructose and glucose the following experiment was carried out. To mimic the product of an isomerization of glucose, an aqueous solution of saccharides was provided, wherein the solution comprised 30% wt of saccharides and wherein the saccharides consisted for 65% wt of glucose and for 35% wt of fructose. To the solution methyl levulinate was added in a quantity of 5% wt, based on the amount of saccharides. A homogeneous solution resulted. Subsequently the solution obtained was subjected to evaporation. During the evaporation of water, white turbidity developed. Water was evaporated to a level of about 8% wt, based on the original amount of water in the aqueous solution, and a composition of fructose and glucose precipitated from the remaining methyl levulinate/water mixture.

1. A process for the preparation of a fructose-rich solution from a solid composition comprising fructose and glucose, comprising:
i) admixing the solid composition comprising fructose and glucose with a selective solvent that consist for at least 80% wt of methanol to obtain a slurry of glucose-rich solids in a methanolic fructose-rich solution; and
ii) separating the methanolic fructose-rich solution from the glucose-rich solids.

2. The process according to claim 1, wherein the selective solvent consists for at least 90% wt of methanol.

3. The process according to claim 1, wherein the selective solvent comprises up to 10% wt of water.

4. The process according to claim 1, wherein the methanolic fructose-rich solution is separated from the glucose-rich solids by means of one or more operations selected from the group consisting of sedimentation, filtration, centrifugation, flotation and decantation.

5. The process according to claim 1, wherein the admixing of the solid composition and the selective solvent is carried out at a temperature in the range of 0 to 50° C.

6. The process according to claim 1, wherein the amount of selective solvent that is admixed with the solid composition is in the range of 0.1:1 to 10:1, based on the weight of selective solvent per weight of solid composition.

7. The process according to claim 1, wherein the methanolic fructose-rich solution has a fructose to glucose weight ratio in the range of 75:25 to 95:5.

8. The process according to claim 1, wherein the solid composition consists for at least 95% wt of fructose and glucose, based on the weight of the solid composition.

9. The process according to claim 1, wherein the solid composition comprises fructose and glucose in a fructose to glucose weight ratio of 1:10 to 10:1.

10. The process according to claim 1, wherein the solid composition has been derived from the isomerization of glucose to fructose.

11. The process according to claim 1, wherein the solid composition has been obtained by the evaporation of water from a liquid saccharide composition comprising water, fructose and glucose.

12. The process according to claim 11, wherein the evaporation has been achieved by mixing an aqueous saccharides solution comprising water, fructose and glucose, with a carrier liquid in which the saccharides are insoluble and that has a boiling point higher than that of water, and subjecting the mixture thus obtained to an evaporation step.

13. The process according to claim 12, wherein the carrier liquid is immiscible with water.

14. The process according to claim 13, wherein the carrier liquid is selected from the group consisting of hydrocarbon oil, polyisobutylene, poly-alpha-olefins and mixtures thereof.

15. The process according to claim 12, wherein the carrier liquid is miscible with water.

16. The process according to claim 15, wherein the carrier liquid is selected from the group of alkylene glycols and alkyl levulinates.

17. The process according to claim 10, wherein at least part of the glucose-rich solids is recycled to the isomerization of glucose to fructose.

18. A method the preparation of the methyl ether of 5-hydroxymethylfurfural wherein at least a part of the methanolic fructose-rich solution prepared according to claim 1 is reacted as starting material with methanol in the presence of a heterogeneous or homogeneous acid catalyst at a temperature in the range of 150 to 300° C.
